(19) United States
(12) Patent Application Publication
(10) Pub. No.: US 2003/0162236 A1
(43) Pub. Date: Aug. 28, 2003

(54) Compensation for variability in specific binding in quantitative assays

(75) Inventors: Paul C. Harris, Bothell, WA (US); Brian G. Richards, British Columbia (CA)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Response Biomedical Corporation, British Columbia (CA)

(21) Appl. No.: 09/817,781

(22) Filed: Mar. 26, 2001

(51) Int. Cl. C12Q 1/70; C12Q 1/68; G01N 33/537; G01N 33/543

(52) U.S. Cl. 435/7.92; 435/5; 435/6

(57) ABSTRACT

Methods for quantitatively measuring the amount of an analyte of interest in a fluid sample are disclosed. The methods involve providing a membrane having an application point, a contact region comprising analyte-binding particles, a sample capture zone, and a control capture zone, where the contact region is between the application point and the sample capture zone, and the sample capture region is between the contact region and the control capture zone. In the assays, a fluid allows transport components of the assay by capillary action through the contact region, to and through the sample capture zone and subsequently to and through the control capture zone. In a “sandwich assay” embodiment, the amount of analyte in the fluid sample is related to a corrected analyte-binding particle amount, which can be determined, for example, as a ratio of the amount of analyte-binding particles in the sample capture zone and the amount of analyte-binding particles in the control capture zone. In a “competitive assay” embodiment, the membrane has an application point, a contact region comprising analyte-coated particles, a sample capture zone, and a control capture zone, where the contact region is between the application point and the sample capture zone, and the sample capture zone is between the contact region and the control capture zone. In this “competitive assay” embodiment, the amount of analyte in the fluid sample is inversely related to a corrected analyte-coated particle amount, which can be determined, for example, as a ratio of the amount of analyte-coated particles in the sample capture zone and the amount of analyte-coated particles in the control capture zone.

### BACKGROUND OF THE INVENTION

Quantitative analysis of cells and analytes in fluid samples, particularly bodily fluid samples, often provides critical diagnostic and treatment information for physicians and patients. Quantitative immunoassays utilize the specificity of the antigen (Ag)-antibody (Ab) reaction to detect and quantitate the amount of an Ag or Ab in a sample. In solid phase immunoassays, one reagent (e.g., the Ag or Ab) is attached to a solid surface, facilitating separation of bound reagents or analytes from free reagents or analytes. The solid phase is exposed to a sample containing the analyte, which binds to its Ag or Ab; the extent of this binding is quantitated to provide a measure of the analyte concentration in the sample. Transduction of the binding event into a measurable signal, however, is affected by a number of interferences, such as variability in binding of components of the assay, which are not associated with the presence or amount of the analyte. These interferences limit the specificity and applicability of quantitative immunoassays.

### SUMMARY OF THE INVENTION

The invention relates to methods of measuring the amount of an analyte of interest in a fluid sample, using a solid phase assay such as a quantitative immunochromatographic assay (e.g., a sandwich immunoassay or an inhibition immunoassay), in which an internal control is used to compensate for variability in specific binding of assay components. In the methods of the invention, an analyte of interest and a capture reagent are used as part of a specific binding pair.

For quantitative immunochromatographic assays, the methods use a membrane strip made of a suitable material, such as cellulose nitrate or glass fiber, which has sufficient porosity and the ability to be wet by the fluid containing the analyte, and which allows movement of particles by capillary action. The membrane strip has an application point, a contact region, a sample capture zone and a control capture zone; the contact region is between the application point and the sample capture zone, and the sample capture zone is between the contact region and the control capture zone.

In a “sandwich” type assay, immobilized in the contact region is a population of analyte-binding particles, such as liposomes or organic polymer latex particles. The analyte-binding particles are coated with a binding agent (e.g., an antibody) to the analyte of interest. The particles can be labeled, using a colorimetric, fluorescent, luminescent, chemiluminescent, enzyme-linked label (e.g., in an ELISA), or other appropriate label, to facilitate detection. A sample capture reagent (e.g., an agent that binds to the analyte of interest, such as an antibody to the analyte of interest) is immobilized in the sample capture zone. A control capture reagent (e.g., an agent that binds to the analyte-binding particles, such as an anti-immunoglobulin antibody) is immobilized in the control capture zone.

In the methods, the application point of the membrane strip is contacted with the fluid sample to be assayed for the analyte of interest. The membrane strip is then maintained under conditions which are sufficient to allow capillary action of fluid to transport the analyte of interest, if analyte is present in the sample, through the membrane strip to and through the contact region. The apparatus is further maintained so that when analyte of interest reaches the contact region, analyte binds to the analyte binding agent coated on the analyte-binding particles immobilized in the contact region. Analyte-binding particles, including those which are bound with analyte (“analyte-bound” particles) are mobilized by fluid and move by capillary action through the strip to and through the sample capture zone.

The sample capture reagent interacts with analyte-bound particles; interaction of the sample capture reagent and the analyte-bound particles results in arrest of analyte-bound particles in the sample capture zone. Capillary action of the fluid further mobilizes the analyte-binding particles not only to and through the sample capture zone, but also to and through the control capture zone, where they bind to the control capture reagent. Capillary action of the fluid continues to mobilize the remaining unbound particles past the control capture zone (e.g., into a wicking pad). The amount of analyte-binding particles that are arrested in the sample capture zone, and in the control capture zone, are then determined.

The amount of analyte of interest in the fluid sample is then determined. For example, the amount of analyte of interest in the fluid sample can be determined as a ratio between 1) the amount of analyte-binding particles that are arrested in the sample capture zone, and 2) the amount of analyte-binding particles in the control capture zone. Alternatively, the amount of analyte of interest in the fluid sample can be determined as a ratio between 1) the amount of analyte-binding particles that are arrested in the sample capture zone, and 2) the sum of the amount of analyte-binding particles in the control capture zone and the amount of analyte-binding particles that are arrested in the sample capture zone.

In an alternative immunochromatographic assay, the fluid sample to be assayed for the analyte of interest is applied directly to the sample capture zone of the apparatus. The membrane strip is maintained under appropriate conditions so that analyte in the fluid sample interacts with the sample capture reagent, and is immobilized in the sample capture zone. Water or an appropriate buffer is then added to the application point of the membrane, to mobilize the analyte-binding particles, which are then moved by capillary action into and through the sample capture zone and subsequently into and through the control capture zone. The membrane strip is further maintained under conditions which allow interaction of the analyte-binding particles with analyte that is immobilized in the sample capture zone. Interaction of the analyte-binding particles with immobilized analyte arrests movement of analyte-bound particles in the sample capture zone; interaction of the analyte-binding particles with the control capture reagent arrests movement of analyte-binding particles in the control capture zone. The amount of analyte in the fluid sample is determined by taking into consideration the amount of analyte-binding particles that are arrested in the control capture zone, as described above.

In another embodiment, in a “competitive” or “inhibition” type immunochromatographic assay, immobilized in the contact region is a population of analyte-coated particles. The particles can be labeled as described above, to facilitate detection. A sample capture reagent (e.g., an agent that binds to the analyte of interest, such as an antibody to the analyte of interest) is immobilized in the sample capture zone. A control capture reagent (e.g., an agent that binds to the analyte-coated particles and not to the analyte itself) is immobilized in the control capture zone.

In the methods, the application point of the membrane strip is contacted with the fluid sample to be assayed for the analyte of interest. The membrane strip is then maintained under conditions which are sufficient to allow capillary action of fluid to transport the analyte of interest, if analyte is present in the sample, through the membrane strip to and through the contact region. The apparatus is further maintained so that when analyte of interest reaches the contact region, analyte-coated particles are mobilized by fluid and move by capillary action, along with any analyte present in the sample, through the strip to and through the sample capture zone.

The sample capture reagent interacts with analyte-coated particles; interaction of the sample capture reagent and the analyte-coated particles results in arrest of analyte-coated particles in the sample capture zone. Because of competition between the analyte-coated particles and analyte (if present) in the sample for binding sites on the sample capture reagent in the sample capture zone, the amount of analyte-coated particles arrested in the sample capture zone is inversely proportional to the amount of analyte in the sample. Capillary action of the fluid further mobilizes the analyte-coated particles not only to and through the sample capture zone, but also to and through the control capture zone, where they bind to the control capture reagent. Capillary action of the fluid continues to mobilize the remaining unbound particles past the control capture zone (e.g., into a wicking pad). The amount of analyte-coated particles that are arrested in the sample capture zone, and in the control capture zone, are then determined.

The amount of analyte of interest in the fluid sample is then determined. For example, the amount of analyte of interest in the fluid sample is inversely related to a ratio between 1) the amount of analyte-coated particles that are arrested in the sample capture zone, and 2) the amount of analyte-coated particles in the control capture zone. Alternatively, the amount of analyte of interest in the fluid sample is inversely related to a ratio between 1) the amount of analyte-coated particles that are arrested in the sample capture zone, and 2) the sum of the amount of analyte-coated particles in the control capture zone and the amount of analyte-coated particles that are arrested in the sample capture zone.

The flow of fluid through a solid phase in such quantitative assays contributes to the dynamic nature of the assays: the amount of binding of analytes to particles, as well as the location of particles in relation to positions on the solid phase, is in flux. Variations in the structure of the solid phase reactants, such as porosity of the solid phase reactants, as well as variations in the viscosity of the fluid sample and other factors, can thereby contribute to variability in specific binding of components of the assays. The methods of the invention compensate for the variations that result from the dynamic nature of the assays, thereby allowing more accurate determination of the amounts of analytes of interest in solutions. Furthermore, the system increases the sensitivity of the assay when a ratio (e.g., the ratio of the amount of analyte-binding particles that are arrested in the sample capture zone, and the amount of analyte-binding particles in the control capture zone; or the ratio of the amount of analyte-coated particles that are arrested in the sample capture zone, and the amount of analyte-coated particles in the control capture zone) is used to determine the amount of an analyte of interest. As more particles are bound at the sample capture zone, fewer are available at the control capture zone, thereby simultaneously decreasing the denominator and increasing the numerator with an increase in concentration of the analyte of interest. In addition, when the ratio is employed, the use of absolute signal levels are canceled out in the calculation of the amount of analyte of interest; thus, inaccuracies in calibration of a signal reader used to detect the signal levels are minimized.

### BRIEF DESCRIPTION OF THE DRAWINGS

FIG. 1 depicts the dynamic nature of a quantitative immunochromatographic assay, in which a fluid containing analyte of interest is added at an application point of the membrane (step 1), and the membrane is incubated such that the fluid mobilizes particles coated with antibody that binds to the analyte of interest from the contact region, and moves them along the membrane (step 2) to the sample capture zone and subsequently to the control capture zone (step 3).

FIGS. 2A-2F are a series of graphs depicting the results of a quantitative immunochromatographic assay measuring the amount of myoglobin in a series of test samples. The amount of signal corresponding to the amount of fluorescent analyte-binding particles detected in the sample capture zone and in the control capture zone, are shown as a function of the amount of myoglobin in the test sample. FIG. 2A, 0 ng/ml myoglobin; FIG. 2B, 2.5 ng/ml myoglobin; FIG. 2C, 3 ng/ml myoglobin; FIG. 2D, 10 ng/ml myoglobin; FIG. 2E, 20 ng/ml myoglobin; FIG. 2F, 40 ng/ml myoglobin.

FIG. 3 is a graph depicting a standard curve for measuring the amount of myoglobin by the “sandwich” quantitative immunochromatographic assay. The ratio (R) of the amount of the analyte-binding particle amount present in the sample capture zone, to the sum of the analyte-binding particle amount present in the control capture zone and the analyte-binding particle amount present in the sample capture zone is compared with the concentration of myoglobin (ng/ml) in the sample.

The foregoing and other objects, features and advantages of the invention will be apparent from the following more particular description of preferred embodiments of the invention, as illustrated in the accompanying drawings in which like reference characters refer to the same parts throughout the different views. The drawings are not necessarily to scale, emphasis instead being placed upon illustrating the principles of the invention.

### DETAILED DESCRIPTION OF THE INVENTION

A description of preferred embodiments of the invention follows.

The current invention pertains to methods of correcting for variability in specific binding of reagents in quantitative, ligand-binding assays. As described herein, Applicants have developed a means for compensating for variability in specific binding in assays, thereby enhancing the accuracy of measurement of the amount of an analyte of interest. The methods involve inclusion, within the assay, of an internal control comprising a control capture reagent, in a control capture zone, that specifically binds to analyte-binding particles. The behavior of the analyte-binding particles with regard to the control capture reagent is used to compensate for the amount of variability in the reaction of the analyte-binding particles with the surfaces of the assay. The amount of variability of the analyte-binding particles can then be taken into consideration in a determination of the amount of analyte of interest, thereby allowing a more accurate determination of the amount of specific reaction of analyte-binding particles. For example, a corrected amount of analyte-binding particles can be determined by use of a ratio between 1) the amount of analyte-binding particles that are arrested in the sample capture zone, and 2) the amount of analyte-binding particles in the control capture zone; or use of a ratio between 1) the amount of analyte-binding particles that are arrested in the sample capture zone, and 2) the sum of the amount of analyte-binding particles in the control capture zone and the amount of analyte-binding particles that are arrested in the sample capture zone; or use of another appropriate calculation to eliminate the variability in the specific binding component of the reaction. The amount of analyte of interest can then be calculated from the corrected amount of analyte-binding particles.

An “assay,” as used herein, refers to an in vitro procedure for analysis of a sample to determine the presence, absence, or quantity of one or more analytes. The ligand-binding assays of the inventions utilize an analyte and an analyte binding agent. The analyte and the analyte binding agent are members of a specific “binding pair,” in which a first member of the binding pair (e.g., analyte) reacts specifically with a second member (e.g., the binding agent). One or both members of the binding pair can be an antibody: for example, a first member of the binding pair (e.g., an analyte of interest) can be an antibody, and a second member of the binding pair (e.g., a binding agent) can be anti-immunoglobulin antibody. Alternatively, the first member of the binding pair (e.g., the analyte) can be an antigen, and the second member of the binding pair (e.g., the binding agent) can be an antibody. In a preferred embodiment, the assay is an “immunoassay” which utilizes antibodies as a component of the procedure. In a particularly preferred embodiment, the immunoassay is a quantitative immunochromatographic assay such as a “sandwich” assay, which is a test for an analyte in which a fluid test sample containing analyte is contacted with a membrane having immobilized on it particles coated with an analyte-binding agent, such as antibodies to the analyte, causing capillary action of components of the system through the membrane, with a positive result indicated by detection of interaction between analyte and binding agent-coated particles in a capture zone of the membrane, the amount of binding agent-coated particles in the capture zone being related to the amount of analyte in the test sample. For representative quantitative immunochromatographic assays, see, for example, U.S. Pat. No. 5,753,517, the entire teachings of which is incorporated by reference herein. In another particularly preferred embodiment, the immunoassay is a quantitative immunochromatographic assay such as an “inhibition” or “competitive” assay, which is a test for an analyte in which a fluid test sample containing analyte is contacted with a membrane having immobilized within it particles coated with the analyte, causing capillary action of components of the system through the membrane, with a positive result indicated by detection of interaction between agent-coated particles in a capture zone of the membrane, the amount of agent-coated particles in the capture zone being inversely related to the amount of analyte in the test sample.

In other embodiments of the assays of the invention, neither the analyte nor the binding agent are antibodies: for example, the first member of the binding pair can be a ligand, and the second member of the binding pair can be a receptor; alternatively, the first member of the binding pair can be a lectin, and the second member of the binding pair can be a sugar. In still another embodiment, the first member of the binding pair can be a nucleic acid (e.g., DNA, RNA), and the second member of the binding pair can be a nucleic acid which specifically hybridizes to the first member of the binding pair. “Specific hybridization,” as used herein, refers to refers to the ability of a first nucleic acid to hybridize to a second nucleic acid in a manner such that the first nucleic acid does not hybridize to any nucleic acid other than to the second nucleic acid (e.g., when the first nucleic acid has a higher similarity to the second nucleic acid than to any other nucleic acid in a sample wherein the hybridization is to be performed). “Stringency conditions” for hybridization is a term of art which refers to the incubation and wash conditions, e.g., conditions of temperature and buffer concentration, which permit hybridization of a particular nucleic acid to a second nucleic acid; the first nucleic acid may be perfectly (i.e., 100%) complementary to the second, or the first and second may share some degree of complementarity which is less than perfect (e.g., 70%, 75%, 80%, 85%, 90%, 95%). For example, certain high stringency conditions can be used which distinguish perfectly complementary nucleic acids from those of less complementarity. “High stringency conditions”, “moderate stringency conditions” and “low stringency conditions” for nucleic acid hybridizations are explained on pages 2.10.1-2.10.16 and pages 6.3.1-6.3.6 in Current Protocols in Molecular Biology (Ausubel, F. M. et al., “Current Protocols in Molecular Biology”, John Wiley & Sons, (1998), the entire teachings of which are incorporated by reference herein). The exact conditions which determine the stringency of hybridization depend not only on ionic strength (e.g., 0.2×SSC, 0.1×SSC), temperature (e.g., room temperature, 42° C., 68° C.) and the concentration of destabilizing agents such as formamide or denaturing agents such as SDS, but also on factors such as the length of the nucleic acid sequence, base composition, percent mismatch between hybridizing sequences and the frequency of occurrence of subsets of that sequence within other non-identical sequences. Thus, equivalent conditions can be determined by varying one or more of these parameters while maintaining a similar degree of identity or similarity between the two nucleic acid molecules.

Regardless of the composition of the analyte and the binding agent, these two components nevertheless form a specific binding pair, in which the first member reacts specifically with the second member. Specific interaction between the members of the binding pair indicates that the first member of the binding pair preferentially binds or otherwise interacts with the second member of the binding pair, preferably to the exclusion of any binding to another compound in the assay.

The terms, “analyte” or “analyte of interest,” as used herein, refer to a first member of a binding pair as described above. The analyte is a molecule or compound for which the amount will be measured. Examples of analytes include proteins, such as hormones or enzymes; glycoproteins; peptides; small molecules; polysaccharides; antibodies; nucleic acids; drugs; toxins (e.g., environmental toxins); viruses or virus particles; portions of a cell wall; and other compounds. In a preferred embodiment, the analyte is “immunogenic,” which indicates that antibodies (as described below) can be raised to the analyte, or to an analyte that is bound to a carrier (e.g., a hapten-carrier conjugate, for which antibodies can be raised to the hapten). In some representative embodiments, the analyte of interest can be myoglobin; CK-MB; troponin I; PSA; digoxin; theophylline; a hormone (e.g., T-3 or T-4); or a drug of abuse (LSD, THC, barbituates, etc.).

The analyte is in a fluid sample. The fluid sample can be a fluid having relatively few components, for example, an aqueous solution containing the analyte of interest; alternatively, the fluid sample can be a fluid having many components, such as a complex environmental sample (e.g., sewage, groundwater), or a complex biological fluid (e.g., whole blood, plasma, serum, urine, cerebrospinal fluid, saliva, semen, vitreous fluid, synovial fluid, or other biological fluid). In one representative embodiment, if the analyte of interest is myoglobin, the fluid sample is usually whole blood, plasma or serum. If desired, the fluid sample can be diluted; for example, if a complex biological fluid is used as the fluid sample, it can be diluted with a solution (e.g., an aqueous solution). Alternatively, if the analyte of interest is not in solution (e.g., the analyte of interest is in a solid sample), it can be extracted into solution; for example, if the analyte of interest is a nucleic acid, it can be extracted from cells of interest into a solution (e.g., an aqueous solution).

The “analyte-binding agent,” as used herein, refers to second member of a binding pair as described above. The analyte-binding agent is a compound that specifically binds to the analyte (the first member of the binding pair), such as an antibody, a hapten or drug conjugate, a receptor, or another binding partner. In a preferred embodiment, the analyte-binding agent is an antibody to the analyte of interest.

“Sandwhich” Assays

In one embodiment of the invention, a quantitative assay such as the quantitative immunochromatographic assay described in U.S. Pat. No. 5,753,517, is performed. In such an assay, a solid phase, such as a rapid antigen measurement platform (RAMP™.) apparatus (U.S. Pat. No. 5,753,517), is used. The solid phase includes a membrane strip having an application point, a contact region, a sample capture zone, and a control capture zone. The solid phase may optionally include a wicking pad following the control capture zone, and a sample pad preceding the application point. The membrane strip can be made of a substance having the following characteristics: sufficient porosity to allow capillary action of fluid along its surface and through its interior; the ability to allow movement of coated particles by capillary action (i.e., it must not block the particles); and the ability to be wet by the fluid containing the analyte (e.g., hydrophilicity for aqueous fluids, hydrophobicity for organic solvents). Hydrophobicity of a membrane can be altered to render the membrane hydrophilic for use with aqueous fluid, by processes such as those described in U.S. Pat. No. 4,340,482, or U.S. Pat. No. 4,618,533, which describe transformation of a hydrophobic surface into a hydrophilic surface. Examples of membrane substances include: cellulose, cellulose nitrate, cellulose acetate, glass fiber, nylon, polyelectrolyte ion exchange membrane, acrylic copolymer/nylon, and polyethersulfone. In a preferred embodiment, the membrane strip is made of cellulose nitrate.

The “application point” is the position on the membrane where a fluid sample is applied. The “contact region” of the membrane is adjacent to the application point. Immobilized (coated on and/or permeated in the membrane) in the “contact region” of the membrane is a population of “analyte-binding particles” which are coated with the analyte-binding agent. The population of particles varies, depending on the size and composition of the particles, the composition of the membrane, and the level of sensitivity of the assay. The population typically ranges approximately between 1×10³ and 1×10⁹, although fewer or more can be used if desired. In a preferred embodiment, the population is approximately 2×10⁷ particles.

The analyte-binding particles are particles which can be coated with the analyte-binding agent (the second member of the binding pair). In a preferred embodiment, the analyte-binding particles are liposomes, organic polymer latex particles, inorganic fluorescent particles or phosphorescent particles. In a particularly preferred embodiment, the particles are polystyrene latex beads, and most particularly, polystyrene latex beads that have been prepared in the absence of surfactant, such as surfactant-free Superactive Uniform Aldehyde/Sulfate Latexes (Interfacial Dynamics Corp., Portland, Oreg.).

The size of the particles is related to porosity of the membrane: the particles must be sufficiently small to be transported along the membrane by capillary action of fluid. The particles can be labeled to facilitate detection. The particles are labeled by a means which does not significantly affect the physical properties of the particles; for example, the particles are labeled internally (that is, the label is included within the particle, such as within the liposome or inside the polystyrene latex bead). Representative labels include luminescent labels; chemiluminescent labels; phosphorescent labels; enzyme-linked labels; and colorimetric labels, such as dyes or fluorescent labels. In one embodiment, a fluorescent label is used. In another embodiment, phosphorescent particles are used, particularly “up-converting” phosphorescent particles, such as those described in U.S. Pat. No. 5,043,265.

The particles are coated with an analyte-binding agent that is a second member of the binding pair. As described above, the analyte-binding agent (second member of the binding pair) specifically and preferentially binds to the analyte of interest (first member of the binding pair). Representative analyte-binding agents include antibodies (or fragments thereof); haptens; drug conjugates; receptors; or other binding partners. In one preferred embodiment, the analyte-binding agent is an antibody to the analyte of interest. Antibodies can be monoclonal antibodies or polyclonal antibodies. The term “antibody”, as used herein, also refers to antibody fragments which are sufficient to bind to the analyte of interest. Alternatively, in another embodiment, molecules which specifically bind to the analyte of interest, such as engineered proteins having analyte binding sites, can also be used (Holliger, P. and H. R. Hoogenbloom, Trends in Biotechnology 13:7-9 (1995); Chamow, S. M. and A. Ashkenazi, Trends in Biotechnology 14:52-60:1996)). In still another embodiment, if the analyte of interest is a drug, a hapten or other drug conjugate can be used as the analyte binding agent. Alternatively, in a further embodiment, a receptor which binds to the analyte can be used (e.g., if the analyte of interest is a ligand). If the analyte is an antibody of known specificity, the particles can be coated with the antigen against which the analyte-antibody is directed, or can be coated with antibody to the analyte-antibody. Furthermore, because the analyte and the analyte binding agent form a binding pair, compounds or molecules described as representative analytes can also serve as analyte binding agents, and those described as representative analyte binding agents can similarly serve as analytes, as described herein.

The contact region of the membrane is between the application point and the “sample capture zone” of the membrane. The sample capture zone refers to a point on the membrane strip at which a “sample capture reagent” is immobilized (e.g., coated on and/or permeated through the membrane). The sample capture reagent is an analyte-binding agent, such as those described above. The sample capture reagent need not be the same analyte binding agent as described above; however, the sample capture reagent also forms a binding pair with the analyte of interest, in that it specifically and preferentially binds to the analyte of interest. In a preferred embodiment, the sample capture reagent is an antibody directed against the analyte; it can be directed against the same epitope of the analyte as, or against a different epitope of the analyte from, the epitope that binds to the antibodies used as analyte-binding agents coated on the particles.

The apparatus additionally includes a “control capture reagent” immobilized in a “control capture zone.” The control capture reagent is a reagent which reacts with the analyte binding particles, but which does not interact with the analyte to be measured: for example, the control capture reagent can react with the analyte-binding agent on the analyte-binding agent-coated particles; with another material on the particles; or with the particles themselves. For example, if the analyte-binding agent is an antibody, the control capture reagent can be an anti-immunoglobulin antibody. In a preferred embodiment, the analyte-binding agent is an antibody, and the control capture reagent is an anti-immunoglobulin antibody. The control capture reagent is immobilized on the membrane (coated on and/or permeated in the membrane) in a control capture zone.

The control capture zone is positioned such that the sample capture zone is between the contact region and the control capture zone. In a preferred embodiment, the control capture zone is closely adjacent to the sample capture zone, so that the dynamics of the capillary action of the components of the assay are similar (e.g., essentially the same) at both the control capture zone and the sample capture zone. Although they are closely adjacent, the control capture zone and the sample capture zone are also sufficiently spaced such that the particles arrested in each zone can be quantitated individually (e.g., without cross-talk). Furthermore, in a preferred embodiment, the sample capture zone is separated from the contact region by a space that is a large distance, relative to the small distance between the sample capture zone and the control capture zone. The speed of the capillary front (the border of the fluid moving through the membrane by capillary action) is inversely related to the distance of the capillary front from the application point of the fluid. Because particle capture is the rate limiting step in the assay, the distance between the contact region (where the capillary front mobilizes analyte-binding particles) and the capture zones (where particles are captured) must be sufficient to retard the speed of the capillary front to a rate that is slow enough to allow capture of particles when the capillary front reaches the sample capture zone. In addition, the distance must be sufficiently large so that the total time of migration (movement of the capillary front through the entire membrane) is long enough to allow free analyte in a fluid sample to bind to analyte-binding particles. The optimal distances between the components on the membrane strip can be determined and adjusted using routine experimentation.

To perform the quantitative immunochromatographic assay, a fluid sample to be assessed for the presence of the analyte of interest, as described above, is used. The fluid can be a fluid that wets the membrane material; that supports a reaction between the analyte of interest and the analyte binding agent, such as the antibody/antigen reaction (i.e., does not interfere with antibody/antigen interaction); and that has a viscosity that is sufficiently low to allow movement of the fluid by capillary action. In a preferred embodiment, the fluid is an aqueous solution (such as a bodily fluid).

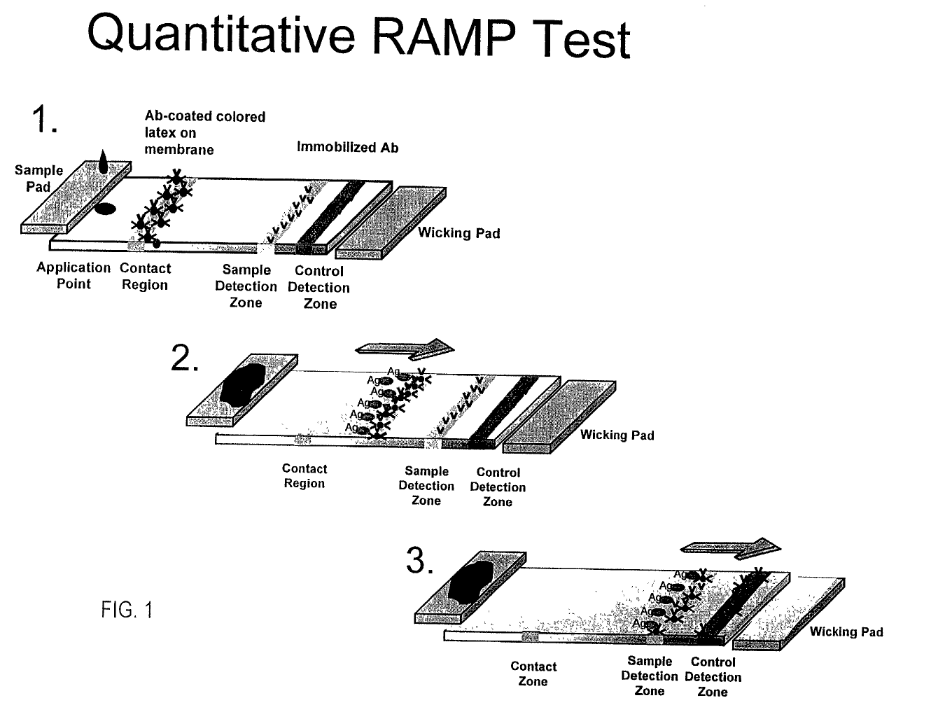

In a first embodiment of the quantitative immunochromatographic assay, the application point of the membrane strip is contacted with the fluid sample to be assayed for the analyte of interest (see FIG. 1, step 1). After the membrane strip is contacted with the fluid sample containing the analyte of interest at the application point, the membrane strip is maintained under conditions which allow fluid to move by capillary action to and through the “contact region” of the membrane, thereby transporting the analyte of interest (if present in the fluid) to and through the contact region. As the analyte is transported to and through the contact region, analyte that is present in the fluid (if any is present) binds to the analyte-binding particles immobilized in the contact region. “Binding” of analyte to the analyte-binding particles indicates that the analyte-binding agent coated onto the particle is interacting with (e.g., binding to) analyte of interest. Analyte-binding particles which have been maintained under conditions allowing analyte in the fluid (if present) to bind to the analyte-binding particles immobilized in the contact region are referred to herein as “contacted analyte-binding particles”. Contacted analyte-binding particles may or may not have analyte bound to the analyte-binding agent, depending on whether or not analyte is present in the fluid sample and whether analyte has bound to the analyte-binding agent on the analyte-binding particles. Because there are multiple binding sites for analyte on the analyte-binding particles, the presence and the concentration of analyte bound to analyte-binding particles varies; the concentration of analyte bound to the analyte-binding particles increases proportionally with the amount of analyte present in the fluid sample, and the probability of an analyte-binding particle being arrested in the sample capture zone (as described below) similarly increases with increasing amount of analyte bound to the analyte-binding particles. Thus, the population of contacted analyte-binding particles may comprise particles having various amount of analyte bound to the analyte-binding agent, as well as particles having no analyte bound to the analyte-binding agent (just as the analyte-binding particles initially have no analyte bound to the analyte-binding agent).

The contacted analyte-binding particles are further mobilized by capillary action of the fluid from the fluid sample (see FIG. 1, step 2), and the contacted analyte-binding particles move along the membrane to and through the “sample capture zone” on the membrane and subsequently to and through the “control capture zone” (see FIG. 1, step 3). The membrane strip is maintained under conditions (e.g., sufficient time and fluid volume) which allow the contacted analyte-binding particles to move by capillary action along the membrane to and through both the sample capture zone and (subsequently) to the control capture zone, and subsequently beyond the control capture zone (e.g., into a wicking pad), thereby removing any non-bound particles from the capture zones.

The movement of some of the contacted analyte-binding particles is arrested by binding of contacted analyte-binding particles to the sample capture reagent in the sample capture zone, and subsequently by binding of some of the contacted analyte-binding particles to the control capture reagent in the control capture zone. In one preferred embodiment in which the analyte-binding agent is antibody to the antigen of interest, the control capture reagent can be antibody against immunoglobulin of the species from which the analyte-binding agent is derived. In this embodiment, the antibody to immunoglobulin should be non-cross reactive with other components of the sample: for example, if a human sample is being tested, an antibody that does not react with human immunoglobulin can be used as the control capture reagent.

Sample capture reagent binds to contacted analyte-binding particles by binding to analyte which is bound to analyte-binding agent on the contacted analyte-binding particles. The term, “sample-reagent-particle complexes”, as used herein, refers to a complex of the sample capture reagent and contacted analyte-binding particles. Contacted analyte-binding particles are arrested in the sample capture zone, forming the sample-reagent-particle complexes, due to capture of contacted analyte-binding particles by interaction of analyte with sample capture reagent in the sample capture zone.

Control capture reagent binds to contacted analyte-binding particles by binding to analyte-binding agent on the contacted analyte-binding particles. The term, “control-reagent-particle complexes,” as used herein, refers to a complex of the control capture reagent and contacted analyte-binding particles. Contacted analyte-binding particles are arrested in the control capture zone, forming the control-reagent-particle complexes, due to capture of contacted analyte-binding particles by interaction of analyte binding particles with control capture reagent in the control capture zone. As indicated above, the control capture reagent interacts with the analyte-binding particles (e.g., with the analyte-binding agent on the analyte-binding agent-coated particles, or another material on the particles, or with the particles themselves), but not with the analyte itself.

Capillary action subsequently moves any contacted analyte-binding particles that have not been arrested in either the sample capture zone or the control capture zone, onwards beyond the control capture zone, thereby removing any particles that have not been arrested from both the sample capture zone and the control capture zone. In a preferred embodiment, the fluid moves any contacted analyte-binding particles that have not been arrested in either capture zone into a wicking pad which follows the control capture zone.

The amount of analyte-binding particles arrested in the sample capture zone is then detected. The analyte-binding particles are detected using an appropriate means for the type of label used on the analyte-binding particles. In a preferred embodiment, the amount of analyte-binding particles is detected by an optical method, such as by measuring the amount of fluorescence of the label of the analyte-binding particles. The amount of analyte-binding particles arrested in the control capture zone is detected in the same manner as the amount of analyte-binding particles in the sample capture zone. In one embodiment, the amount of analyte-binding particles is represented by a curve that is directly related to the amount of label present at positions along the solid phase (e.g., the membrane strip). For example, the amount of particles at each position on the membrane strip (e.g., at the sample capture zone and the control capture zone, and/or areas in between or adjacent to the sample capture zone and the control capture zone, and/or other areas of the membrane strip) can be determined and plotted as a function of the distance of the position along the membrane strip. The amount of particles can then be calculated as a function of the area under the curve, which is related to the amount of label present.

A corrected analyte-binding particle amount is determined, and the amount of analyte can then be determined from the corrected analyte-binding particle amount using appropriate calculation. The corrected analyte-binding particle amount is based on the amount of analyte-binding particles arrested in the sample capture zone and in the control capture zone. For example, in one embodiment, the corrected analyte-binding particle amount is determined as a ratio (R) of the analyte-binding particle amount present in the sample capture zone to the analyte-binding particle amount present in the control capture zone. The amount of analyte present can be then determined from the corrected analyte-binding particle amount (the ratio), utilizing a standard curve. The standard curve is generated by preparing a series of control samples, containing known concentrations of the analyte of interest in the fluid in which the analyte is to be detected (such as serum depleted of the analyte). The quantitative immunochromatographic assay is then performed on the series of control samples; the value of R is measured for each control sample; and the R values are plotted as a function of the concentration of analyte included in the control sample. Samples containing an unknown amount of analyte (the “test samples”) are assayed by measuring the value of R for the test sample, and the concentration of analyte in the test sample is determined by referring to the standard curve. As above, one standard curve can be generated and used for all test samples in a lot (e.g., for all test samples using a specified preparation of test reagents); it is not necessary that the standard curve be re-generated for each test sample. In another embodiment, the corrected analyte-binding particle amount is determined as a ratio (R) of the amount of the analyte-binding particle amount present in the sample capture zone, to the sum of the analyte-binding particle amount present in the control capture zone and the analyte-binding particle amount present in the sample capture zone. The amount of analyte present can be then determined from corrected analyte-binding particle amount (the ratio), utilizing a standard curve. Alternatively, other ratios and/or standard curves can also be used to determine the amount of analyte in the sample. In addition, if desired, the amount of label that is present in the background can be subtracted from the analyte-binding particle amount present in the sample capture zone and the analyte-binding particle amount present in the control capture zone prior to calculation of the ratio (R).

In a second embodiment of the invention, the capture zone of the membrane strip, rather than the application point, is contacted with the fluid sample. The membrane strip is maintained under conditions which are sufficient to allow binding of analyte of interest in the fluid sample to the sample capture reagent in the sample capture zone, thereby generating arrested analyte. Subsequently, the application point of the membrane is contacted with water or a buffer. The buffer can be an aqueous fluid that wets the membrane material; that supports a reaction between the analyte of interest and the analyte-binding agent (e.g., does not interfere with antibody/antigen interaction); and that has a viscosity that is sufficiently low to allow movement of the fluid by capillary action. Examples of buffers include, for example, saline, or 50 mM Tris-HCl, pH 7.4. The buffer mobilizes and transports the population of analyte-binding particles immobilized in the membrane at the contact region by capillary action to and through the sample capture zone and subsequently to and through the control capture zone. The membrane strip is further maintained under conditions which are sufficient to allow interaction of the arrested analyte (arrested in the sample capture zone) with the analyte-binding particles. Interaction of arrested analyte with analyte-binding particles arrests the movement of the analyte-binding particles, and generates arrested sample-reagent-particle complexes. The amount of analyte-binding particles in the sample capture zone is then measured, as described above, as is the amount of analyte-binding particles arrested in the control capture zone, and the amount of analyte in the fluid sample is determined by determining the amount of corrected analyte-binding particles, as described above. For example, the amount of analyte of interest in the fluid sample can be related to the corrected analyte-binding particle amount (e.g., by a standard curve). If desired, the amount can also be determined using additional internal control components, and determining ratios, as described above.

“Competitive” or “Inhibition” Assays

In another embodiment of the invention, a quantitative assay, such as the quantitative immunochromatographic assay described in U.S. Pat. No. 5,753,517, is performed as a competitive or inhibition assay. In such an assay, a solid phase, such as a rapid antigen measurement platform (RAMP™) apparatus (U.S. Pat. No. 5,753,517), is used. The membrane strip, made of a substance as described above, includes an application point, a contact region, a sample capture zone, and a control capture zone. The membrane strip may optionally include a wicking pad following the control capture zone, and a sample pad preceding the application point. As before, the “application point” is the position on the membrane where a fluid sample is applied. The “contact region” of the membrane is adjacent to the application point. Immobilized in the “contact region” of the membrane is a population of particles, as described above, which are coated with the analyte of interest (in lieu of being coated with an analyte binding agent, as described for the “sandwich” assays) or with an analog of the analyte of interest. An “analog” of the analyte, as used herein, is a compound that has similar binding characteristics as the analyte, in that is forms a binding pair with the analyte-binding agent as described above. The analyte or analog of the analyte can be coated directly on the particles, or can be indirectly bound to the particles. As used below, the term “analyte-coated particles” can refer to particles that are coated either with analyte of interest or with an analog of the analyte of interest.

The contact region of the membrane is between the application point and the sample capture zone of the membrane, at which the sample capture reagent is arrested. The sample capture reagent is an analyte-binding agent, such as those described above (e.g., a second member of a binding pair). In a preferred embodiment, the sample capture reagent is an antibody directed against the analyte.

The apparatus additionally includes a control capture reagent immobilized in a control capture zone which is positioned such that the sample capture zone is between the contact region and the control capture zone. As above, the control capture reagent reacts with the analyte binding particles, but does not interact with the analyte to be measured: for example, the control capture reagent can react with another material on the particles (e.g., a carrier for the analyte that is bound to the particles; an antibody); or with the particles themselves. In a preferred embodiment, the sample capture reagent and the control capture agent are both antibodies. The control capture reagent is immobilized on the membrane (coated on and/or permeated in the membrane) in the control capture zone.

The components of the competitive assay are positioned in a similar manner as described above with regard to the “sandwich” assay. For example, in a preferred embodiment, the control capture zone is closely adjacent to the sample capture zone, so that the dynamics of the capillary action of the components of the assay are similar (e.g., essentially the same) at both the control capture zone and the sample capture zone; and yet the control capture zone and the sample capture zone are also sufficiently spaced such that the particles arrested in each zone can be quantitated individually. Furthermore, in a preferred embodiment, the sample capture zone is separated from the contact region by a space that is a large distance, relative to the small distance between the sample capture zone and the control capture zone, in order to ensure that the speed of the capillary front is sufficiently slow to allow capture of particles, and the total time of migration is sufficiently long to allow for binding of analyte to the sample capture reagent.

To perform the competitive, quantitative immunochromatographic assay, a fluid sample to be assessed for the presence of the analyte of interest is obtained, as above. The application point of the membrane strip is contacted with the fluid sample to be assayed for the analyte of interest. After the membrane strip is contacted with the fluid sample containing the analyte of interest at the application point, the membrane strip is maintained under conditions which allow fluid to move by capillary action to and through the contact region of the membrane, thereby transporting the analyte of interest (if present in the fluid) to and through the contact region. The analyte-coated particles in the contact region, together with analyte (if present) in the sample, are further mobilized by capillary action of the fluid from the fluid sample, and the analyte-coated particles move along the membrane with the fluid and analyte to and through the “sample capture zone” on the membrane and subsequently to and through the “control capture zone.” The membrane strip is maintained under conditions (e.g., sufficient time and fluid volume) which allow the analyte-coated particles to move by capillary action along the membrane to and through both the sample capture zone and (subsequently) to and through the control capture zone, and subsequently beyond the control capture zone (e.g., into a wicking pad), thereby removing any non-bound particles from the capture zones.

The movement of some of the analyte-coated particles is arrested by binding of analyte-coated particles to the sample capture reagent in the sample capture zone, and subsequently by binding of some of the analyte-coated particles to the control capture reagent in the control capture zone. The analyte-coated particles compete with analyte (if present) in the sample for binding to the sample capture reagent. The sample capture reagent binds to analyte-coated particles by binding to analyte on the analyte-coated particles. The term, “sample-reagent-analyte-coated-particle complexes”, as used herein, refers to a complex of the sample capture reagent and analyte-coated particles. The analyte-coated particles are arrested in the sample capture zone, forming the sample-reagent-analyte-coated-particle complexes, due to capture of the analyte-coated particles by interaction of the analyte on the particles with the sample capture reagent in the sample capture zone.

The control capture reagent binds to analyte-coated particles by binding to any component of the analyte-coated particles except the analyte itself. The term, “control-reagent-analyte-coated particle complexes,” as used above, refers to a complex of the control capture reagent and analyte-coated particles. As above, the analyte-coated particles are arrested in the control capture zone, forming the control-reagent-analyte-coated particle complexes, due to capture of the analyte-coated particles by interaction of the analyte binding particles with the control capture reagent in the control capture zone.

Capillary action subsequently moves any analyte-coated particles that have not been arrested in either the sample capture zone or the control capture zone, onwards beyond the control capture zone, thereby removing any particles that have not been arrested from both the sample capture zone and the control capture zone. In a preferred embodiment, the fluid moves any contacted analyte-coated particles that have not been arrested in either capture zone into a wicking pad which follows the control capture zone.

The amount of analyte-binding particles arrested in the sample capture zone is then detected. The analyte-binding particles are detected using an appropriate means for the type of label used on the analyte-binding particles. In a preferred embodiment, the amount of analyte-binding particles is detected by an optical method, such as by measuring the amount of fluorescence of the label of the analyte-binding particles. The amount of analyte-binding particles arrested in the control capture zone is detected in the same manner as the amount of analyte-binding particles in the sample capture zone. In one embodiment, as described above, the amount of analyte-binding particles is represented by a curve that is directly related to the amount of label present at positions along the solid phase (e.g., the membrane strip). For example, the amount of particles at each position on the membrane strip (e.g., at the sample capture zone and the control capture zone, and/or areas in between or adjacent to the sample capture zone and the control capture zone, and/or other areas of the membrane strip) can be determined and plotted as a function of the distance of the position along the membrane strip. The amount of particles can then be calculated as a function of the area under the curve, which is related to the amount of label present.

A corrected analyte-coated particle amount is determined, and the amount of analyte can then be determined from the corrected analyte-coated particle amount using appropriate calculation. The corrected analyte-coated particle amount is based on the amount of analyte-coated particles arrested in the sample capture zone and in the control capture zone. For example, in one embodiment, the corrected analyte-coated particle amount is inversely proportional to a ratio (R) of the analyte-coated particle amount present in the sample capture zone to the analyte-coated particle amount present in the control capture zone. The amount of analyte present can be then determined from the corrected analyte-coated particle amount (the ratio), utilizing a standard curve. The standard curve is generated by preparing a series of control samples, containing known concentrations of the analyte of interest in the fluid in which the analyte is to be detected (such as serum depleted of the analyte). The quantitative immunochromatographic assay is then performed on the series of control samples; the value of R is measured for each control sample; and the R values are plotted as a function of the concentration of analyte included in the control sample. Samples containing an unknown amount of analyte (the “test samples”) are assayed by measuring the value of R for the test sample, and the concentration of analyte in the test sample is determined by referring to the standard curve. As above, one standard curve can be generated and used for all test samples in a lot (e.g., for all test samples using a specified preparation of test reagents); it is not necessary that the standard curve be re-generated for each test sample. In another embodiment, the corrected analyte-coated particle amount is inversely proportional to a ratio (R) of the amount of the analyte-coated particle amount present in the sample capture zone, to the sum of the analyte-coated particle amount present in the control capture zone and the analyte-coated particle amount present in the sample capture zone. The amount of analyte present can be then determined from corrected analyte-coated particle amount (the ratio), utilizing a standard curve. Alternatively, other ratios and/or standard curves can also be used to determine the amount of analyte in the sample. In addition, if desired, the amount of label that is present in the background can be subtracted from the analyte-binding particle amount present in the sample capture zone and the analyte-binding particle amount present in the control capture zone prior to calculation of the ratio (R).

Although the assays of the invention have been described particularly in relation to quantitative immunochromatographic assays, the assays can similarly be used with other binding pairs as described above (e.g., nucleic acids, receptor-ligands, lectin-sugars), using the same methods as described above with the desired components as the analyte and the and the analyte-binding agent. The invention also includes kits for use in the methods described herein. Kit components can include: first and/or second members of a specific binding pair, buffers, fluid collection means, and control samples for generation of a standard curve; analyte-binding particles and/or control particles, capture reagents, and/or antibodies.

The present invention is illustrated by the following Exemplification, which is not intended to be limiting in any way.

Exemplification Sandwich Assay for Myoglobin

Latex particles of approximately 0.3 microns in diameter (Interfacial Dynamics, Portland, Oreg.) were obtained and dyed using a fluorescent dye that intercalated into the particles (Molecular Probes, Eugene Oreg., or Duke Scientific, Palo Alto, Calif.). Dyed latex particles were coupled to analyte-binding antibodies as follows: particles were washed by centrifugation and resuspended in phosphate buffer at a concentration of approximately 0.2% solids. The antibody (mouse antibody to myoglobin) was prepared to a concentration of 1 mg/ml; 0.5 ml of a 2% latex particle suspension was then added to 4 ml of antibody solution and allowed to incubate with a solution of sodium cyanoborohydride and skim milk, which caused covalent linkage of the antibodies to the particles and saturated the remaining surfaces of the particles with the skim milk protein. The suspension was then vortexed and sonicated to disrupt any aggregates.

Membrane strips were prepared using nitrocellulose membranes (Sartorius). The sample capture agent and the control capture agent were immobilized on the membrane strip in the sample capture zone and the control capture zone, respectively, using a linear striping apparatus (IVEK). For an assay for myoglobin, a goat anti-myoglobin polyclonal antibody (1 mg/ml) was used as the sample capture agent, and a goat anti-mouse immunoglobulin (0.4 mg/ml) was used as the control capture agent. The membrane strips were then allowed to dry.

The membrane strips were blocked by soaking them in a 1% solution of polyvinyl alcohol (PVA) to prevent additional protein binding. The membrane strips were then rinsed in water and dried.

The analyte-binding particles were then applied to the membrane strips at the contact region. First, the contact region is striped with a 30% sucrose solution and allowed to dry. Subsequently the particles were applied as a 0.1% suspension at a striping rate of 2 μl/cm. The membrane was then allowed to dry before performing the assay.

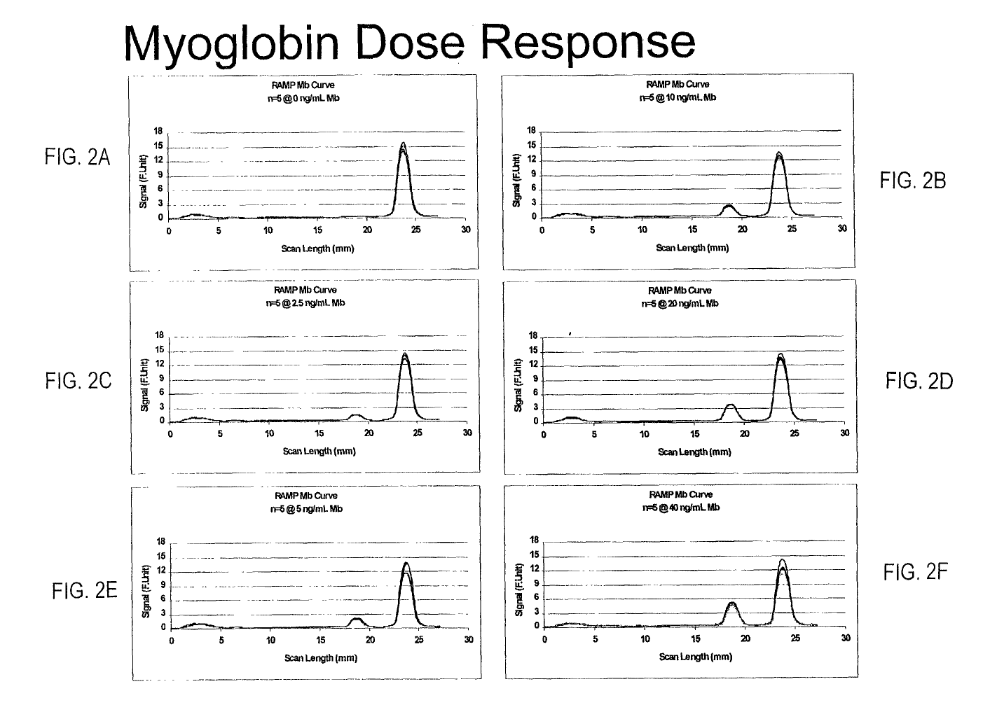

To perform the assay, a sample of a serial dilution of buffer containing myoglobin (0 ng/ml; 2.5 ng/ml; 5 ng/ml; 10 ng/ml; 20 ng/ml; 40 ng/ml) was added to a membrane strip at the application point, and the membrane strips were then maintained at room temperature while the fluid moved through the membrane strip by capillary action. Subsequently, the amount of contacted analyte-binding particles was measured in the sample capture zone and in the control capture zone by detecting the amount of fluorescence. Results are shown in FIGS. 2A-2F, where it can be seen that the area under the curve (depicting the amount of fluorescence) which is present just before the 20 mm of the scan length (the position of the sample capture zone) increases with increasing concentration of myoglobin, whereas the area under the curve which is present just before the 25 mm of the scan length (the position of the control capture zone) remains approximately constant. The area under the curve varies because of test to test variability (e.g., the area under the curve for the control capture zone varies by the same percent as does the area under the curve for the sample capture zone); this variability is corrected for by the methods described herein.

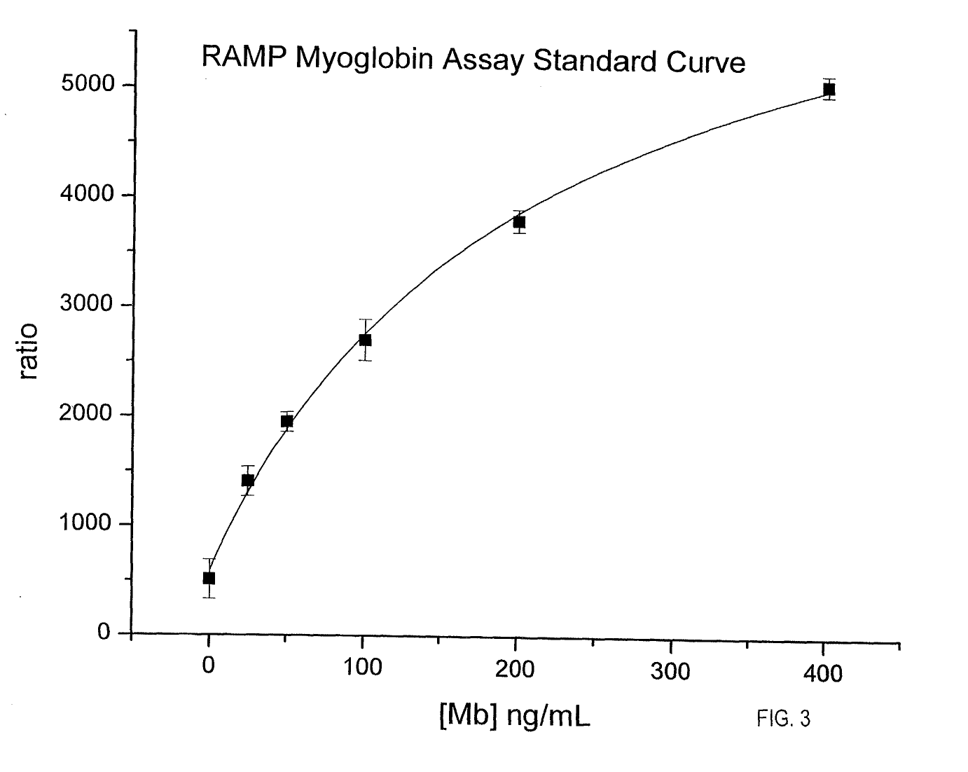

A standard curve (FIG. 3) was generated from the data. The ratio (R) of the amount of the analyte-binding particle amount present in the sample capture zone (calculated by integrating the area under the curve at the sample capture zone), to the sum of the analyte-binding particle amount present in the control capture zone (calculated by integrating the area under the curve at the control capture zone) and the analyte-binding particle amount present in the sample capture zone (calculated as described above), was determined and compared with the concentration of myoglobin (ng/ml). It can be seen that the ratio increases with increasing concentration of myoglobin.

While this invention has been particularly shown and described with references to preferred embodiments thereof, it will be understood by those skilled in the art that various changes in form and details may be made therein without departing from the scope of the invention encompassed by the appended claims.

What is claimed is:

1. A method for quantitatively measuring the amount of an analyte of interest in a fluid sample, comprising:
a) providing a membrane strip comprising an application point, a contact region, a sample capture zone and a control capture zone, wherein the contact region is between the application point and the sample capture zone and the sample capture zone is between the contact region and the control capture zone;
b) contacting the application point of the membrane strip with the fluid sample to be assayed for the analyte of interest;
c) maintaining the membrane strip under conditions which allow fluid to transport analyte of interest in the fluid sample by capillary action through the strip to and through the contact region, the contact region having a population of analyte-binding particles immobilized therein, wherein the analyte-binding particles are coated with an analyte-binding agent;
d) further maintaining the membrane strip under conditions which allow analyte of interest, if present in the sample, to bind to analyte-binding particles, thereby generating contacted analyte-binding particles; allow the fluid in the sample to mobilize and transport contacted analyte-binding particles by capillary action through the strip to and through the sample capture zone, the sample capture zone having a sample capture reagent immobilized thereon; and allow contacted analyte-binding particles to bind to the sample capture reagent;
e) further maintaining the membrane strip under conditions which allow the fluid in the sample to transport contacted analyte-binding particles by capillary action through the strip to and through the control capture zone, the control capture zone having a control capture reagent immobilized thereon; and allow contacted analyte-binding particles to bind to the control capture reagent;
f) further maintaining the membrane strip under conditions which allow the fluid in the sample to transport any contacted analyte-binding particles not bound to the sample capture reagent or to the control capture reagent by capillary action beyond the control capture zone;
g) determining the amount of contacted analyte-binding particles in the sample capture zone and the amount of contacted analyte-binding particles in the control capture zone;
h) determining a corrected analyte-binding particle amount from the amount of analyte-binding particles in the sample capture zone and the amount of analyte-binding particles in the control capture zone,
wherein the amount of analyte of interest in the fluid sample is directly related to the corrected analyte-binding particle amount.

2. The method of claim 1, wherein the corrected analyte-binding particle amount is determined as a ratio of the amount of analyte-binding, particles in the sample capture zone, to the amount of analyte-binding particles in the control capture zone.

3. The method of claim 1, wherein the corrected analyte-binding particle amount is determined as a ratio of the amount of analyte-binding particles in the sample capture zone, to the sum of the amount of analyte-binding particles in the control capture zone and the amount of analyte-binding particles in the sample capture zone.

4. The method of claim 1, wherein the membrane strip is made of cellulose nitrate or glass fiber.

5. The method of claim 1, wherein the particles are latex beads.

6. The method of claim 1, wherein the particles are labeled.

7. The method of claim 6, wherein the label is selected from the group consisting of: calorimetric, fluorescent, phosphorescent, luminescent, chemiluminescent, and enzyme-linked molecule.

8. The method of claim 1, wherein the analyte and the analyte-binding agent are members of a binding pair, and one member of the binding pair is selected from the group consisting of: a protein, a hormone, an enzyme, a glycoprotein, a peptide, a small molecule, a polysaccharide, a lectin, an antibody, an antibody fragment, a nucleic acid, a drug, a drug conjugate, a toxin, a virus, a virus particle, a portion of a cell wall, a hapten, and a receptor.

9. The method of claim 1, wherein the analyte-binding agent is selected from the group consisting of: an antibody; an antibody fragment; a hapten; a drug conjugate; and a receptor.

10. The method of claim 9, wherein the analyte-binding agent is an antibody.

11. The method of claim 10, wherein the sample capture reagent is an antibody selected from the group consisting of: an antibody directed against the same epitope as the antibody on the analyte-binding particles, and an antibody directed against a different epitope as the antibody on the analyte-binding particles.

12. The method of claim 10, wherein the control capture reagent is an anti-immunoglobulin antibody.

13. The method of claim 1, wherein the test sample is selected from the group consisting of: whole blood, plasma, serum, urine, cerebrospinal fluid, saliva, semen, vitreous fluid, or synovial fluid.

14. The method of claim 1, wherein the analyte of interest is selected from the group consisting of: myoglobin, CK-MB, troponin I, and PSA.

15. The method of claim 1, wherein in step (f) the fluid in the sample transports any contacted analyte-binding particles not bound to the sample capture reagent or to the control capture reagent by capillary action beyond the control capture zone into a wicking pad.

16. A method for measuring the amount of an analyte of interest in a fluid sample, comprising:
a) providing a membrane strip comprising an application point, a contact region, a sample capture zone and a control capture zone, wherein the contact region is between the application point and the sample capture zone and the sample capture zone is between the contact region and the control capture zone;
b) contacting the sample capture zone of the membrane strip with the fluid sample, the sample capture zone having a sample capture reagent immobilized thereon, and maintaining the membrane strip under conditions which allow analyte of interest, if present in the sample, to bind to the sample capture reagent in the sample capture zone, thereby generating arrested analyte;
c) contacting the application point of the membrane strip with a buffer;
d) maintaining the membrane strip under conditions which allow the buffer to mobilize and transport a population of analyte-binding particles immobilized in the contact region by capillary action to and through the sample capture zone, wherein the analyte-binding particles are coated with an antibody to the analyte; and allow the arrested analyte to interact with analyte-binding particles, thereby generating arrested analyte-particle complexes;
e) further maintaining the membrane strip under conditions which allow the buffer to transport analyte-binding particles by capillary action to and through the control capture zone, the control capture zone having a control capture reagent immobilized thereon; and allow analyte-binding particles to bind to the control capture reagent;
f) further maintaining the membrane strip under conditions which allow the fluid in the sample to transport any analyte-binding particles not bound to the sample capture reagent or to the control capture reagent by capillary action beyond the control capture zone;
g) determining the amount of analyte-binding particles in the sample capture zone and the amount of analyte-binding particles in the in the control capture zone; and
h) determining a corrected analyte-binding particle amount from the amount of analyte-binding particles in the sample capture zone and the amount of analyte-binding particles in the control capture zone,
wherein the amount of analyte of interest in the fluid sample is directly related to the corrected analyte-binding particle amount.

17. The method of claim 16, wherein the corrected analyte-binding particle amount is determined as a ratio of the amount of analyte-binding particles in the sample capture zone, to the amount of analyte-binding particles in the control capture zone.

18. The method of claim 16, wherein the corrected analyte-binding particle amount is determined as a ratio of the amount of analyte-binding particles in the sample capture zone, to the sum of the amount of analyte-binding particles in the control capture zone and the amount of analyte-binding particles in the sample capture zone.

19. A method for quantitatively measuring the amount of an analyte of interest in a fluid sample, comprising:
a) providing a membrane strip comprising an application point, a contact region, a sample capture zone and a control capture zone, wherein the contact region is between the application point and the sample capture zone and the sample capture zone is between the contact region and the control capture zone;
b) contacting the application point of the membrane strip with the fluid sample to be assayed for the analyte of interest;
c) maintaining the membrane strip under conditions which allow fluid to transport analyte of interest in the fluid sample by capillary action through the strip to and through the contact region, the contact region having a population of analyte-coated particles immobilized therein, wherein the analyte-coated particles are coated with analyte of interest;
d) further maintaining the membrane strip under conditions which allow the fluid in the sample to mobilize and transport analyte-coated particles by capillary action through the strip to and through the sample capture zone, the sample capture zone having a sample capture reagent immobilized thereon; and allow analyte-coated particles to bind to the sample capture reagent;
e) further maintaining the membrane strip under conditions which allow the fluid in the sample to transport analyte-coated particles by capillary action through the strip to and through the control capture zone, the control capture zone having a control capture reagent immobilized thereon; and allow analyte-coated particles to bind to the control capture reagent;
f) further maintaining the membrane strip under conditions which allow the fluid in the sample to transport any analyte-coated particles not bound to the sample capture reagent or to the control capture reagent by capillary action beyond the control capture zone;
g) determining the amount of analyte-coated particles in the sample capture zone and the amount of analyte-coated particles in the control capture zone;
h) determining a corrected analyte-coated particle amount from the amount of analyte-coated particles in the sample capture zone and the amount of analyte-coated particles in the control capture zone,
wherein the amount of analyte of interest in the fluid sample is inversely related to the corrected analyte-coated particle amount.

20. The method of claim 19, wherein the corrected analyte-coated particle amount is determined as a ratio of the amount of analyte-coated particles in the sample capture zone, to the amount of analyte-coated particles in the control capture zone.

21. The method of claim 19, wherein the corrected analyte-coated particle amount is determined as a ratio of the amount of analyte-coated particles in the sample capture zone, to the sum of the amount of analyte-coated particles in the control capture zone and the amount of analyte-coated particles in the sample capture zone.

22. The method of claim 19, wherein the membrane strip is made of cellulose nitrate or glass fiber.

23. The method of claim 19, wherein the particles are latex beads.

24. The method of claim 19, wherein the particles are labeled.

25. The method of claim 24, wherein the label is selected from the group consisting of: calorimetric, fluorescent, phosphorescent, luminescent, chemiluminescent, and enzyme-linked molecule.

26. The method of claim 19, wherein the analyte and the analyte-binding agent are members of a binding pair, and one member of the binding pair is selected from the group consisting of: a protein, a hormone, an enzyme, a glycoprotein, a peptide, a small molecule, a polysaccharide, a lectin, an antibody, an antibody fragment, a nucleic acid, a drug, a drug conjugate, a toxin, a virus, a virus particle, a portion of a cell wall, a hapten, and a receptor.

27. The method of claim 19, wherein the analyte-binding agent is selected from the group consisting of: an antibody; an antibody fragment; a hapten; a drug conjugate; and a receptor.

28. The method of claim 27, wherein the analyte-binding agent is an antibody.

29. The method of claim 28, wherein the control capture reagent is an antibody.

30. The method of claim 19, wherein the test sample is selected from the group consisting of: whole blood, plasma, serum, urine, cerebrospinal fluid, saliva, semen, vitreous fluid, or synovial fluid.

31. The method of claim 19, wherein the analyte of interest is selected from the group consisting of: digoxin, theophylline, hormone T-3, hormone T-4, LSD, THC, and a barbiturate.

32. The method of claim 19, wherein in step (f) the fluid in the sample transports any analyte-coated particles not bound to the sample capture reagent or to the control capture reagent by capillary action beyond the control capture zone into a wicking pad.
